(19) United States
(12) Patent Application Publication
(10) Pub. No.: US 2026/0047944 A1
(43) Pub. Date: Feb. 19, 2026

(54) ELECTRONIC PROSTHETIC HAND THAT OPERATED BY DETECTING HAND MOVEMENTS

(71) Applicant: MAND.RO CO., LTD., Bucheon-si (KR)

(72) Inventor: Sang Ho YI, Seoul (KR)

(21) Appl. No.: 19/004,393

(22) Filed: Dec. 29, 2024

(30) Foreign Application Priority Data
Aug. 13, 2024 (KR) 10-2024-0108634

(51) Int. Cl. A61F 2/58; A61F 2/70

(52) U.S. Cl. CPC A61F 2/586; A61F 2/70; A61F 2002/704

(57) ABSTRACT

Provided is an electronic prosthetic hand that detects and operates hand movements. The electronic prosthetic hand may include a prosthetic hand including a finger unit capable of joint movement and a control unit that controls the finger unit, a first sensor module positioned on a user's finger, a second sensor module positioned on the back of the user's hand, and a gyro sensor provided in each of the first and second sensor modules to measure displacement according to a change in the positions of the first and second sensor modules. The electronic prosthetic hand can be used more precisely because the first and second sensor modules detect the movements of the user's finger and back of the hand, respectively, and implement joint movements of the finger unit based on the displacement difference values according to the movements of the finger and back of the hand.

### CROSS-REFERENCE TO RELATED APPLICATION

This application claims priority to and the benefit of Korean Patent Application No. 10-2024-0108634, filed on Aug. 13, 2024, the disclosure of which is incorporated herein by reference in its entirety.

### TECHNICAL FIELD

The present invention relates to an electronic prosthetic hand. More specifically, the present invention relates to an electronic prosthetic hand that can detect and operate hand movements through a gyro sensor or the like.

### BACKGROUND ART

In modern society, the number of amputees is increasing due to a number of complex factors, including the increase in chronic diseases, trauma and infections, aging, lifestyle factors, and medical accidents. The market for various medical devices, such as prosthetic hand, is growing as a means to replace the bodies of these amputees.

Early prosthetic hands mainly provided basic functions such as supplementing appearance or supporting body. However, in modern times, the majority of prosthetic hands are electronic prosthetic hands that implement joint movement using strings or hooks, or motors to implement joint movement. These electronic prosthetic hands receive signals from the user and perform various movements by including electronic components such as electromyography sensors, motors, and batteries. They detect electrical signals generated from the user's muscles, interpret them, and perform joint movements using electric motors. These electronic prosthetic hands receive signals from the user and perform various movements by including electronic components such as electromyography sensors, motors, and batteries. They detect electrical signals generated from the user's muscles, interpret them, and perform joint movements using electric motors.

Electronic prosthetic hands can be adjusted and used to meet a variety of daily activities and occupational needs because they respond to the user's muscle signals to implement natural movements. Moreover, they have the advantage of superior gripping power compared to conventional prosthetic hands because they enable joint movement using motors.

Meanwhile, when using an electromyography sensor, there is a disadvantage of time delay in controlling the prosthetic hand by detecting the electric signal of the muscle. In addition, when using an electronic prosthetic hand for a long time, there is a problem that the responsiveness of the prosthetic hand decreases as the signal strength weakens due to muscle fatigue. There is also a problem that the electromyography signal of the electronic prosthetic hand may become unstable depending on the surrounding electromagnetic interference or skin condition. Furthermore, since the amputation location and shape are different for each amputee, there is a problem that mass production is difficult and the manufacturing cost increases when using an electromyography sensor for an electronic prosthetic hand. Therefore, in order to solve the above-mentioned problems, the development of an electronic prosthetic hand that is easy to purchase and use and can increase the reliability of the product is required.

### DISCLOSURE

### Technical Problem

The present invention is directed to solve the problem of time delay in controlling a prosthetic hand by detecting an electric signal of a muscle using an electromyography sensor.

The present invention is directed to solve the problem of reduced operation reliability of a prosthetic hand due to instability of an electromyography signal when using an electromyography sensor.

The present invention is directed to providing an electronic prosthetic hand that reduces the manufacturing cost and enables mass production.

### Technical Solution

The present invention provides an electronic prosthetic hand including a finger unit capable of joint movement and a control unit controlling the finger unit, a first sensor module positioned on a user's finger, a second sensor module positioned on the back of the user's hand, and a gyro sensor provided in each of the first sensor module and the second sensor module, and characterized in that the finger unit moves jointly when a displacement difference value between a first displacement according to a change in the position of the first sensor module and a second displacement according to a change in the position of the second sensor module occurs.

In addition, the finger unit can move jointly when the displacement difference value is greater than a first correction threshold value stored in advance.

In addition, the finger unit can move jointly when the size of the second displacement is smaller than a second correction threshold value stored in advance.

In addition, the second sensor module is used as a reference coordinate, and when the first displacement of the first sensor module is directed toward the user's palm, the finger unit can perform a joint movement of bending, and when directed toward the back of the user's hand, the finger unit can perform a joint movement of extending. In addition, the first sensor module and the second sensor module each further include an acceleration sensor that measures acceleration of the first sensor module and the second sensor module, and the finger portion can move the joint at a speed corresponding to the acceleration of the first sensor module measured by the acceleration sensor.

In addition, the first sensor module can be connected to the control unit through wireless communication.

In addition, the first sensor module and the second sensor module can be IMU sensors.

### Advantageous Effects

According to the present invention, the electronic prosthetic hand has excellent operation reliability because the first sensor module and the second sensor module respectively detect the movement of the user's fingers and the back of the hand, and implement joint movement.

According to the present invention, since the gyro sensor and the acceleration sensor are used, the manufacturing cost of the electronic prosthetic hand can be reduced.

According to the present invention, the electronic prosthetic hand can be used without being limited to the amputation position or shape, and thus can be applied to various amputation patients.

### DESCRIPTION OF DRAWINGS

FIG. 1 is a schematic diagram illustrating an electronic prosthetic hand according to one embodiment of the present invention.

FIG. 2 is a diagram illustrating a first displacement of a first sensor module and a second displacement of a second sensor module according to one embodiment of the present invention.

FIG. 3 is a diagram illustrating a side view of a finger unit performing a bending joint movement according to one embodiment of the present invention.

FIG. 4 is a diagram illustrating a side view of a finger unit performing an extension joint movement according to one embodiment of the present invention.

FIG. 5 is a diagram illustrating a process of a finger unit performing a joint movement according to the first embodiment of the present invention.

FIG. 6 is a diagram illustrating a process of a finger unit performing a joint movement according to the second embodiment of the present invention.

### MODES OF THE INVENTION

The purposes and effects of the present invention will become clearer through the following detailed description, but the purposes and effects of the present invention are not limited to the following description. In addition, in describing the present invention, the detailed descriptions of well-known technologies related to the present invention that unnecessarily obscure the gist of the present invention will be omitted.

Hereinafter, embodiments of the present invention will be described in detail with reference to the accompanying drawings in order for those skilled in the art to easily perform the present invention. The present invention may be implemented in several different forms and is not limited to the embodiments described herein. In addition, portions irrelevant to the description of the present invention are omitted in the drawings in order to clearly describe the present invention, and the same or similar components are denoted by the same reference numerals.

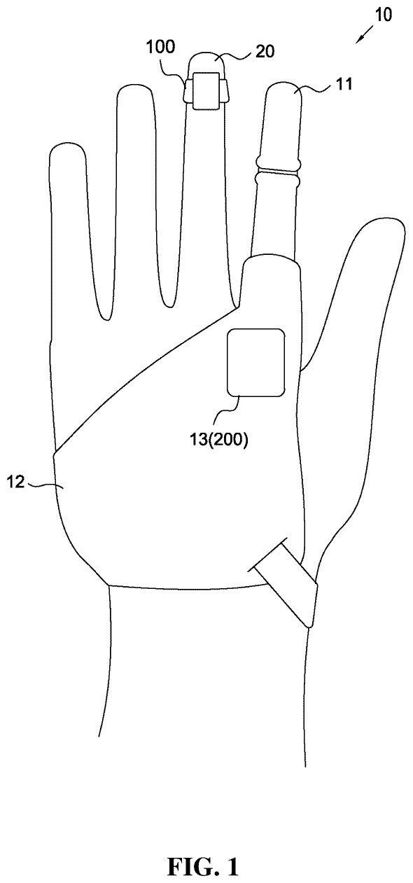

Referring to FIG. 1, an electronic prosthetic hand (10) that detects and operates hand movements according to one embodiment of the present invention is characterized by detecting the movement of a first sensor module (100) worn on a user's finger (20) and causing the finger unit (11) to operate like the first sensor module (100). To this end, the electronic prosthetic hand (10) may include a first sensor module (100) worn on a user's finger (20), a finger unit (11) positioned on the user's affected area, a second sensor module (200) positioned on the back of the user's hand, etc. and supplementing the operational reliability of the first sensor module (100), and a control unit (13) that receives signals from the first sensor module (100) and the second sensor module (200) and transmits an operation execution command to the finger unit (11).

In the present invention, the entire finger (20) is described as being the affected area, and one finger unit (11) is positioned on the affected area of the user. However, the number of finger units (11), their positions, shapes, and structures, etc. may be implemented in various ways, such as the location or number of affected areas.

In addition, if the entire right hand of the user is the affected area, the finger unit (11) is positioned on the right hand, and the first sensor module (100) and the second sensor module (200) may be implemented to be positioned on the user's normal left hand. Also, the control unit (13) may be positioned anywhere. In the present invention, it is sufficient if only the feature of the method of driving the finger unit (11) through the first sensor module (100) and the second sensor module (200) can be implemented.

For example, if a part of a finger (20) and the back of the hand or the palm of the hand are affected areas, the first sensor module (100) may be positioned on the user's finger (20) that can be used, and if the second sensor module (200) cannot be positioned on the back of the hand or the palm of the user, it may be provided in various locations, such as the wrist. However, as described as a reference in the present invention, it is most preferable for the second sensor module (200) to be positioned on the back of the hand or the palm for implementation safety, and in particular, considering hand movements, it is most preferable to be positioned on the back of the hand.

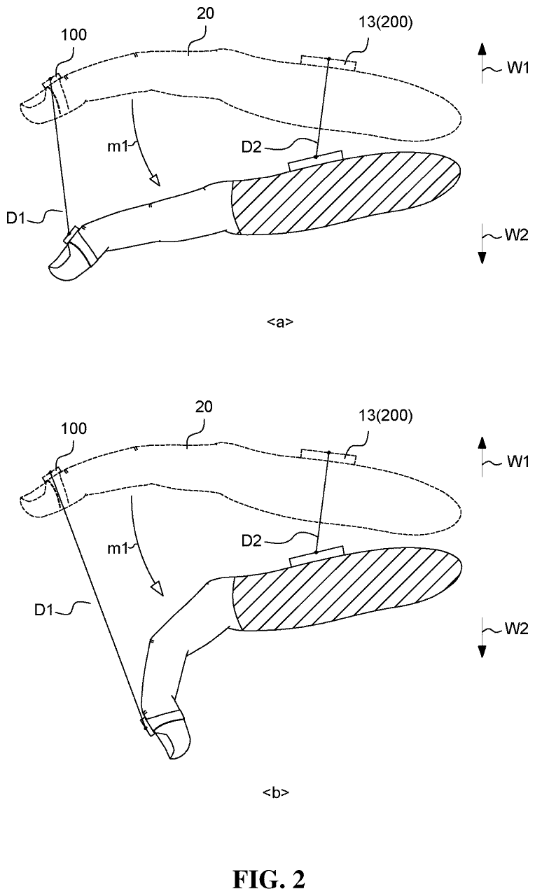

Referring to FIGS. 1 to 4, the finger unit (11) is an electrically operated prosthetic hand that is positioned on the affected area of the user and replaces the finger (20). For this purpose, the finger unit (11) may include a plurality of joints that are not foreign to the user's finger (20) depending on the affected area, and may be provided with gears or hinges (H) connecting each joint. In addition, the finger unit (11) may have a gearbox for reduction built into or external to it, and a power source such as a motor for driving it may be provided internally or externally. The finger unit (11) is capable of joint movement (M2) similar to a human finger (20) according to a signal from the control unit (13), and may perform a bending joint movement (M2) or an extension joint movement (M2).

The finger unit (11) may be provided with a wearing means (12) that helps to wear the device so that it can be positioned on the patient's affected area. The wearing means (12) may be provided in various shapes and sizes depending on the type or location of the user's affected area. In this specification, it is assumed that one finger (20) is the affected area, and an example in which the wearing means (12) is provided in a form that wraps around the back of the hand and the palm so that the finger unit (11) can be fixed to the affected area is described, but the shape or size, etc. are not limited thereto.

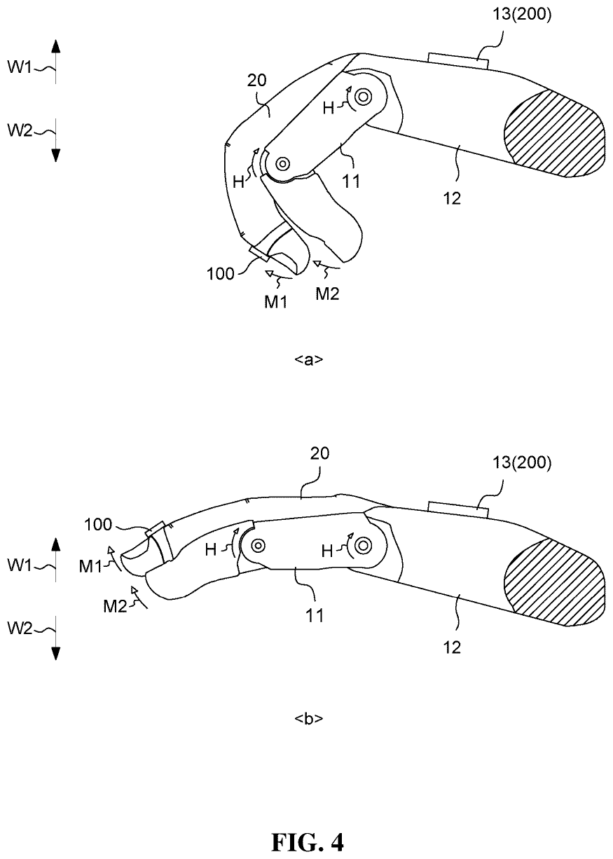

Referring to FIG. 1 and FIG. 4, the electronic prosthetic hand (10) further comprises a first sensor module (100) that can detect the normal movement of a user's finger (20) for joint movement (M2) of a finger unit (11).

The first sensor module (100) can be positioned and worn on the user's finger (20) in various forms, such as a ring or a thimble. The first sensor module (100) can include a gyro sensor and can measure the first displacement (D1), which is a distance and direction value of the first sensor module (100) moving, through the gyro sensor. Therefore, since the first sensor module (100) must measure the first displacement (D1) through the gyro sensor, it is most preferable to wear it on the tip of the finger (20). However, in order for the finger unit (11) to perform more precise movements, the first sensor module (100) may be provided in multiple units and positioned at various locations, such as the tip of the finger (20) and the middle joint of the finger (20), thereby enabling more precise movements.

In addition, if the entire arm is affected, not just the fingers, the second sensor module (200) may be positioned in the direction of the elbow joint, and the first sensor module (100) may be positioned in the direction of the fingers or the back of the hand, so as to control the hand based on the elbow. That is, the present invention only satisfies the technical idea of operating the prosthetic hand based on the measured displacement values of the first sensor module (100) and the second sensor module (200) depending on the location or type of the affected area, so the fingers may be the entire hand depending on the location or type of the affected area of the patient, and may be applied to various affected areas such as the legs.

The finger unit (11) performs joint movement (M2) based on the first displacement (D1) value measured through the first sensor module (100). At this time, even if the user moves the entire arm or does not perform the finger movement (M1) of bending the finger (20), the first displacement (D1) value is generated, which may cause malfunction in such cases. To prevent this, the electronic prosthetic hand (10) may further comprise a second sensor module (200).

The second sensor module (200) is configured to prevent malfunction in advance when the finger unit (11) moves with the first displacement (D1) value measured by the first sensor module (100) and to increase the operational reliability. The second sensor module (200) may also include a gyro sensor like the first sensor module (100). The second sensor module (200) is equipped with a gyro sensor and measures the second displacement (D2), which is a distance and direction value in which the second sensor module (200) moves. The first displacement (D1) measured by the first sensor module (100) and the second displacement (D2) measured by the second sensor module (200) are transmitted to the control unit (13).

The control unit (13) can transmit a command to operate the joint movement (M2) of the finger unit (11) when a difference value between the first displacement (D1) and the second displacement (D2) transmitted from the first sensor module (100) and the second sensor module (200) occurs. Specifically, the control unit (13) can compare the first displacement (D1) and the second displacement (D2) with each other, and can operate the joint movement (M2) of the finger unit (11) when the first displacement (D1) is greater than the second displacement (D2). At this time, it is preferable that the measured first displacement (D1) and the second displacement (D2) are compared with each other based on the same reference time, but is not limited thereto.

Specifically, if the finger unit (11) is subjected to joint movement (M2) based only on the first displacement (D1), the first displacement (D1) is measured when the user moves the finger (20), but the first displacement (D1) is also measured when the entire hand or the entire arm is moved. Therefore, if the finger unit (11) is subjected to joint movement (M2) based only on the first displacement (D1), the finger unit (11) may be subjected to joint movement (M2) even in situations where the user does not intend, which may lower the operation reliability. Therefore, the reliability of the operation may be increased by considering the second displacement (D2) measured from the second sensor module (200) located on the back of the hand.

In the case of the second sensor module (200), since it is located on the back of the hand, if not only the finger (20) moves but the entire arm moves, the values of the first displacement (D1) and the second displacement (D2) may be the same or have only a slight difference. In this case, the control unit (13) determines that the entire arm moves, and can control the finger unit (11) not to move based on the determination result. On the other hand, if the value of the first displacement (D1) is greater than that of the second displacement (D2), the control unit (13) can determine that the finger (20) moves because the change in the position of the finger (20) is greater than the change in the position of the back of the hand. In this case, the control unit (13) can operate the finger unit (11). That is, the control unit (13) compares the first displacement (D1) measured from the first sensor module (100) with the second displacement (D2) measured from the second sensor module (200), and if the first displacement (D1) is greater than the second displacement (D2), the finger unit (11) can perform joint movement (M2).

Meanwhile, as described above, if the control unit (13) determines the operation of the finger unit (11) only by the difference between the first displacement (D1) and the second displacement (D2), whether the finger unit operates can be determined by a small difference. Therefore, in order to increase the reliability of the operation, the control unit (13) can include a pre-specified correction threshold value. At this time, the electronic prosthetic hand (10) can be implemented to transmit the joint movement (M) command of the finger unit (11) according to the first embodiment and the second embodiment according to the configuration of the correction threshold value included in the control unit (13).

In detail, the electronic prosthetic hand (10) according to the first embodiment of the present invention may include a control unit (13) that includes a first correction threshold value. The control unit (13) may compare the difference between the first displacement (D1) and the second displacement (D2) with the first correction threshold value before transmitting a joint movement (M2) command to the finger unit (11), even when the first displacement (D1) is greater than the second displacement (D2). The control unit (13) may transmit the joint movement (M2) command to the finger unit (11) when the difference between the first displacement and the second displacement is greater than the first correction threshold value.

That is, when the first displacement (D1) is greater than the second displacement (D2), the control unit (13) can calculate the difference value and perform the joint movement (M2) of the finger unit (11) only when the difference value is greater than the first correction threshold value stored in advance.

The electronic prosthetic hand (10) according to the second embodiment of the present invention has the same configuration as the first embodiment described above, but can be implemented so that the control unit (13) further includes a second correction threshold value. Specifically, the control unit (13) includes a first correction threshold value and a second correction threshold value specified in advance, and can sequentially compare the received first displacement (D1) and second displacement (D2) with the first correction threshold value and the second correction threshold value to transmit a joint movement (M2) command of the finger unit (11).

Specifically, first, the control unit (13) compares the second displacement (D2) with the second compensation threshold value, and if the second displacement (D2) is smaller than the second compensation threshold value, the first displacement (D1) is compared with the second displacement (D2). The second compensation threshold value is configured to prevent the finger unit (11) from unintentionally performing joint movement (M2) when the entire arm moves during the user's daily life, such as while walking. That is, in order to perform the joint movement (M2) of the finger unit (11), the user can restrict the movement of the back of the hand where the second sensor module (200) is located, maintain the size of the second displacement (D2) smaller than the first compensation threshold value, and induce the joint movement (M2) of the finger unit (11) through the finger movement (M1) of bending the finger (20) in the palm direction (W2) or extending it in the back of the hand direction (W1).

As described above, when the size of the second displacement (D2) is smaller than the second compensation threshold value, the control unit (13) compares the first displacement (D1) and the second displacement (D2), calculates the difference value when the first displacement (D1) is larger than the second displacement (D2), and performs the joint movement (M2) of the finger unit (11) only when the difference value is larger than the first compensation threshold value stored in advance.

As an additional example of the compensation threshold, when examining the joint positions of a human hand, the second sensor module (200) is positioned close to the wrist joint, and the first sensor module (100) is positioned at the tip of the finger (20) in the far direction. Accordingly, when the wrist is bent while the fingers (20) are fully extended with respect to the wrist joint, the second displacement (D2) of the second sensor module (200) close to the wrist joint is small, and the first displacement (D1) of the first sensor module (100) in the far direction from the wrist is large. Even in this case, since the finger movement (M1) was not performed, the finger unit (11) should not perform the joint movement (M2), and therefore, the necessity of the compensation threshold exists.

As an additional example, the first sensor module (100) and the second sensor module (200) may each further include an acceleration sensor for measuring acceleration. The acceleration sensor may measure acceleration during a process in which the positions of the first sensor module (100) and the second sensor module (200) change, and transmit the measured acceleration to the control unit (13). The acceleration measured by the acceleration sensor may be transmitted to the finger unit (11) to control the speed or torque (force) of the joint movement (M2) of the finger unit (11).

More specifically, the acceleration measured from the acceleration sensor is divided into the first acceleration measured by the first sensor module (100) and the second acceleration measured by the second sensor module (200), and it is most preferable that the joint movement (M2) speed of the finger unit (11) be operated based on the difference value obtained by subtracting the second acceleration from the first acceleration.

Meanwhile, the first sensor module (100) and the second sensor module (200) may be IMU (Inertial Measurement Unit) sensors including a gyro sensor and an acceleration sensor.

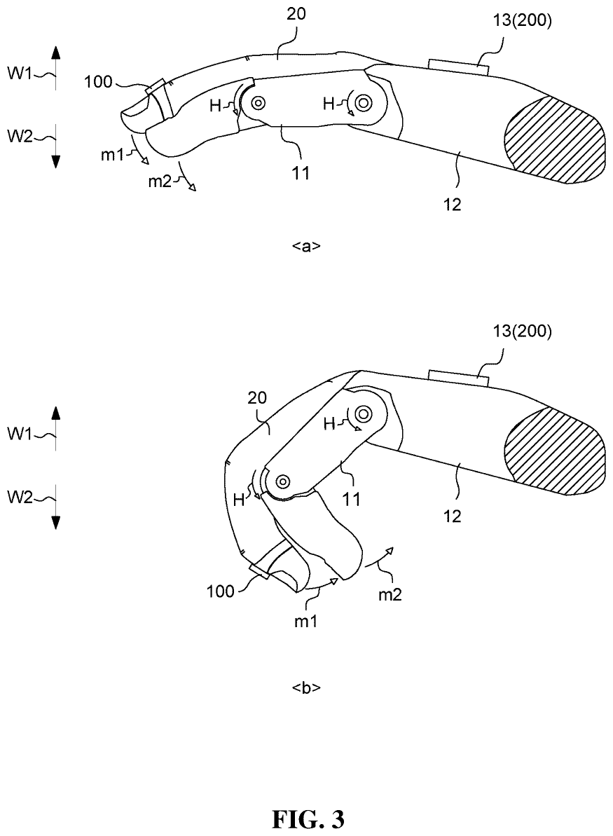

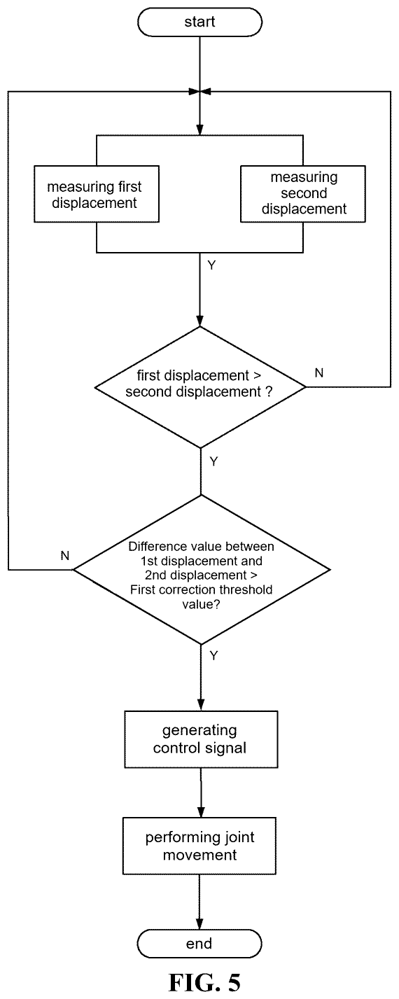

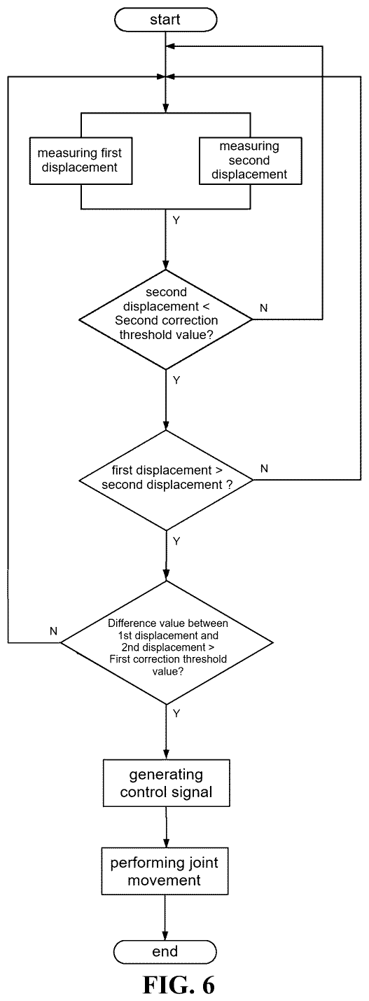

Based on the configuration described above, the operation of the finger unit (11) will be explained as a series of processes as follows. FIG. 5 and FIG. 6 are flowcharts showing a series of processes, FIG. 3 is a side view showing a motion of bending a finger, and FIG. 4 is a side view showing a motion of extending a finger.

When a user performs a finger movement (M1) in which the finger (20) is bent toward the palm (W2) while the finger is extended, the first sensor module (100) located at the tip of the finger (20) can measure a first displacement (D1) along the direction and distance in which the finger is moved.

The control unit (13) receives the measured first displacement (D1) and second displacement (D2), compares the first displacement (D1) and second displacement (D2), and transmits a joint movement (M2) command to the finger unit (11) if the first displacement (D1) is greater than the second displacement (D2). If the finger (20) performs a finger movement (M1) to bend toward the palm direction (W2), the control unit (13) considers the direction and transmits a joint movement (M2) command to bend toward the palm direction (W2). At this time, in order to prevent malfunction in advance, if the first displacement (D1) is greater than the second displacement (D2), the bending joint movement (M2) command is transmitted only when the difference value is greater than a first correction threshold value that is stored in advance.

On the other hand, when the finger (20) and the finger unit (11) are bent and the finger (20) performs a finger movement (M1) in the direction of the back of the hand (W1), the control unit (13) compares the first displacement (D1) and the second displacement (D2), and if the difference is greater than the first correction threshold value stored in advance, the finger unit (11) performs a joint movement (M2) in which the finger is extended.

Meanwhile, when the control unit (13) includes both the first correction threshold value and the second correction threshold value, the joint movement (M2) can be performed only when the second displacement (D2) is less than the second correction threshold value, as in FIG. 6. That is, when the size of the second displacement (D2) is less than the second correction threshold value, the control unit (13) compares the first displacement (D1) and the second displacement (D2), calculates the difference value when the first displacement (D1) is greater than the second displacement (D2), and performs the joint movement (M2) of the finger unit (11) only when the difference value is greater than the first correction threshold value that has been stored in advance.

When performing the bending or extending finger movement (M1) as described above, the control unit (13) can consider the acceleration measured from the acceleration sensor of the first sensor module (100) and the second sensor module (200). In this case, the control unit (13) can consider the bending or extending speed or force of the finger unit (11) when controlling the speed or force of the joint movement (M2) of the finger unit (11).

The above-described exemplary embodiments of the present invention are disclosed to exemplify the present invention and may be variously changed, modified, and added by those skilled in the art within the spirit and scope of the present invention, and such changes, modifications, and additions will fall within the range of the scope. In addition, since such changes, modifications, and additions may be made by those skilled in the art without departing from the technical spirit of the present invention, the present invention is not limited to the above-described description and the accompanying drawings.

In the above-described exemplary system, the methods have described based on a flowchart as a series of steps or blocks, however, the present invention is not limited to the order of the steps, and some steps may occur simultaneously or in an order and steps which are different from those described above. In addition, those skilled in the art may understand that the steps described in the flowchart are not exclusive, other steps may be included, or one or more steps in the flowchart may be deleted without affecting the scope of the present invention.

1. An electronic prosthetic hand including a finger unit capable of joint movement and a control unit for controlling the finger unit, comprising:
a first sensor module positioned on a finger of a user;
a second sensor module positioned on a back of a hand of the user; and
a gyro sensor provided in each of the first sensor module and the second sensor module to measure displacement,
wherein the finger unit performs joint movement when a difference value occurs between a first displacement due to a change in a position of the first sensor module and a second displacement due to a change in a position of the second sensor module.

2. The electronic prosthetic hand of claim 1, wherein the finger unit performs joint movement when the difference value is greater than a first correction threshold value stored in advance.

3. The electronic prosthetic hand of claim 1, wherein the finger unit performs joint movement when the second displacement is less than a second correction threshold value stored in advance.

4. The electronic prosthetic hand of claim 1, wherein the finger unit performs a bending joint movement when the first displacement of the first sensor module with respect to the second sensor module is directed toward a palm of the user,
wherein the finger unit performs an extension joint movement when the first displacement of the first sensor module with respect to the second sensor module is directed toward the user's back of hand direction.

5. The electronic prosthetic hand of claim 1, further comprising an acceleration sensor which is provided in each of the first sensor module and the second sensor module to measure the acceleration of the first sensor module and the second sensor module,
wherein the finger unit performs joint movement at a speed corresponding to the acceleration of the first sensor module measured by the acceleration sensor.

6. The electronic prosthetic hand of claim 1, wherein the first sensor module is wirelessly connected to the control unit.

7. The electronic prosthetic hand of claim 1, wherein the first sensor module and the second sensor module are IMU sensors.

8. The electronic prosthetic hand of claim 5, wherein the first sensor module and the second sensor module are IMU sensors.
